(19) United States
(12) Patent Application Publication
(10) Pub. No.: US 2026/0229361 A1
(43) Pub. Date: Aug. 6, 2026

(54) TUMOR DYNAMICS MODELING USING OMICS-BASED DATA

(71) Applicant: Genentech, Inc., South San Francisco, CA (US)

(72) Inventors: James LU, South San Francisco, CA (US); Omid BAZGIR, South San Francisco, CA (US)

(21) Appl. No.: 19/631,319

(22) Filed: Mar. 27, 2026

Related U.S. Application Data
(63) Continuation of application No. PCT/US24/49345, filed on Sep. 30, 2024.
(60) Provisional application No. 63/586,451, filed on Sep. 29, 2023.
(60) Provisional application No. 63/683,153, filed on Aug. 14, 2024.

(51) Int. Cl. G16H 50/20

(52) U.S. Cl. CPC G16H 50/20

(57) ABSTRACT

Systems and methods of prediction of future tumor data. A tumor embedding at a neural ordinary differential equations (ODE) system is received. The tumor embedding is generated using a tumor encoder and observed tumor data that is observed over a selected period of time after a reference point in time. A graph embedding at the neural ODE is received. The graph embedding fuses drug information, disease information, and gene relationship information. The graph embedding is generated using a heterogenous graph encoder that fuses together at least two or more embeddings generated using bipartite graph convolution attention networks. A predicted tumor size at a future point in time is generated using the tumor embedding, the graph embedding, and the neural ODE system. A candidate tumor treatment is administered to a subject in response to the predicted tumor size being less than a selected threshold.

### CROSS-REFERENCE TO RELATED APPLICATIONS

This application is a continuation of International Application No. PCT/US2024/049345, filed Sep. 30, 2024, which is related to and claims the benefit of the priority date of U.S. Provisional Patent Application No. 63/586,451 filed Sep. 29, 2023, entitled “Tumor Dynamic Modeling Using RNA Sequencing Data,” as well as of U.S. Provisional Patent Application No. 63/683,153 filed Aug. 14, 2024, entitled “Tumor Dynamic Modeling Using RNA Sequencing Data,” each of which is incorporated herein by reference in its entirety.

### TECHNICAL FIELD

The subject matter described herein relates generally to tumor dynamics modeling and more specifically to analysis methods and systems that use deep learning techniques for tumor dynamics modeling using observed tumor data and omics-based data based on the associations between genes, drugs, and diseases.

### BACKGROUND

Understanding how tumors change over time is key to developing proper treatment methods and to determining the success of the treatment method. In particular, understanding how a specific patient's tumor will change over time can help create specific treatment protocols for the specific patient.

Tumor dynamics models are models that can be used to predict how a tumor will change over time. For example, some current methodologies use machine learning models to predict how a specific patient's tumor will change over time with respect to one or more factors. Such factors can include, for example, a tumor's organ of origin, early tumor size data, the drug targets associated with a given treatment(s), a predicted and/or resulting therapeutic response to the given treatment(s), or a combination thereof. But these types of models may be unable to account for sequencing data (e.g., RNA sequencing data) and the complex relationship between sequencing data and the factors described above. Accordingly, these types of models may be less accurate than desired in predicting tumor change over time. Thus, improved methods and systems are needed for modeling tumor dynamics that recognize, take into account, and/or address one or more of the issues described above.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a block diagram of a tumor dynamics prediction system in accordance with one or more example embodiments.

FIG. 2 is a block diagram of the heterogeneous graph encoder from FIG. 1 described in greater detail in accordance with one or more embodiments.

FIG. 3 is a flowchart illustrating an embodiment of a process for predicting tumor dynamics in accordance with one or more embodiments.

FIG. 4 is a flowchart illustrating a process for generating a tumor embedding in accordance with one or more embodiments.

FIG. 5 is a flowchart illustrating a process for generating a graph embedding in accordance with one or more embodiments.

FIGS. 6A and 6B together provide a graphical comparison of tumor volume trace fitting using conventional technology to tumor volume trace fitting using the tumor dynamics prediction system described herein in accordance with one or more example embodiments.

FIG. 7 is an illustration of four different graphs comparing the performance of a tumor dynamics prediction system when a heterogeneous graph encoder is used and when the heterogeneous graph encoder is not used in accordance with one or more embodiments.

FIG. 8 depicts a block diagram illustrating an example of a computing system, in accordance with one or more example embodiments.

When practical, similar reference numbers denote similar structures, features, or elements.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### SUMMARY

In one or more embodiments, a method of predicting future tumor data is provided. A tumor embedding at a neural ordinary differential equations (ODE) system is received. A graph embedding at the neural ODE system is received, wherein the graph embedding fuses drug information, disease information, and gene relationship information. A predicted tumor size at a future point in time is generated using the tumor embedding, the graph embedding, and the neural ODE system. A candidate tumor treatment is administered to a subject in response to the predicted tumor size being less than a selected threshold.

In one or more embodiments, a method of predicting future tumor data is provided. A tumor embedding is generated using a tumor encoder and observed tumor data. A graph embedding is generated using a heterogenous graph encoder and a heterogenous graph that includes nodes in a gene domain, a drug domain, and a disease domain. A predicted tumor size at a future point in time is generated using the tumor embedding, the graph embedding, and a neural ordinary differential equations (ODE) system. A treatment regimen for a treatment to be administered to a subject is adjusted based on the predicted tumor size.

In one or more embodiments, a system comprises one or more data processors and a non-transitory computer readable storage medium containing instructions which, when executed on the one or more data processors, cause the one or more data processors to receive a tumor embedding at a neural ordinary differential equations (ODE) system, receive a graph embedding at the neural ODE system, wherein the graph embedding fuses drug information, disease information, and gene relationship information; generate a predicted tumor size at a future point in time using the tumor embedding, the graph embedding, and the neural ODE system; and administer a candidate tumor treatment to a subject in response to the predicted tumor size being less than a selected threshold.

In one or more embodiments, a system comprises one or more data processors and a non-transitory computer readable storage medium containing instructions which, when executed on the one or more data processors, cause the one or more data processors to generate a tumor embedding using a tumor encoder and observed tumor data; generate a graph embedding using a heterogeneous graph encoder and a heterogeneous graph that includes nodes in a gene domain, a drug domain, and a disease domain; generate a predicted tumor size at a future point in time using the tumor embedding, the graph embedding, and a neural ordinary differential equations (ODE) system; and adjust a treatment regimen for a treatment to be administered to a subject based on the predicted tumor size.

In one or more embodiments, a system comprises one or more data processors and a non-transitory computer readable storage medium containing instructions which, when executed on the one or more data processors, cause the one or more data processors to perform part or all of one or more methods disclosed herein or a portion thereof.

In one or more embodiments, a computer-program product tangibly embodied in a non-transitory machine-readable storage medium, including instructions configured to cause one or more data processors to perform part or all of one or more methods disclosed herein or a portion thereof.

### DETAILED DESCRIPTION

### I. Overview

The embodiments described herein recognize and take into account that omics data can greatly impact how a tumor will grow or shrink over time. But at least some currently available methodologies for predicting tumor dynamics are unable to account for such genomics data or the complex relationship(s) between this genomics data and other factors that affect tumor dynamics including, but not limited to, a tumor's organ of origin, early tumor size data, the drug targets associated with a given treatment(s), a predicted and/or resulting therapeutic response to the given treatment(s), or a combination thereof. For example, a tumor dynamics model that uses only early tumor volume data to predict future tumor data (e.g., future tumor volume data, volume of tumor growth or tumor shrinkage) may have less predictive power as compared to a tumor dynamics model that is able to account for both early tumor volume data and genomics data. However, existing tumor dynamics models may be unable to account for genomics data because combining and/or integrating low-dimensional data, such as early tumor size data, with high-dimensional data, such as omics-based data (e.g., RNA sequence data) that integrates multi-modal drug, disease, and genomics/transcriptomics (e.g., RNA sequence data) is challenging.

Thus, the embodiments described herein provide one or more methods and systems for modeling tumor dynamics in a manner that accounts for high-dimensional omics-based data (e.g., integrated RNA sequence data, drug data, and disease data) in addition to one or more other factors (e.g., a tumor's organ of origin, early tumor size data). The omics-based data may include, for example, drug data that indicates which genes are targeted by different drugs, as well as disease data that indicates which genes are associated with which diseases. The high-dimensional omics-based data may be combined with low-dimensional data (e.g., early tumor size data) to enable improved predictions of tumor response to treatment (e.g., changes in tumor size over time or at a future point in time). The models described herein that take into account both the high-dimensional and low-dimensional data allow future tumor metrics (e.g., tumor size or tumor volume) to be more accurately and reliably predicted, which may be important to, for example, developing and/or adjusting a treatment regimen (e.g., treatment dosages, treatment schedule, treatment protocol) for a patient, predicting a patient's therapeutic response to a given treatment regimen, or both.

Thus, the embodiments described herein enable generation of a tumor dynamics model that is specific for melding high-dimensional data (e.g., high-dimensional omics-based data) with low-dimensional data (e.g., early observed tumor size data) and optionally, other data, for use in modeling (e.g., via a neural ordinary differential equations (ODE) system). Further, the embodiments described herein enable generation of a patient-specific or patient-customized data set that includes data derived from both longitudinal data (e.g., early tumor size) and genomics data (e.g., RNA sequence data) for the tumor dynamics model such that predicting tumor change over time using the tumor dynamics model with the customized data set improves the predictive power and accuracy of the model's output.

In some cases, the data and corresponding predictions may be patient-specific because tumor cells and/or tissue from the patient are engrafted into one or more mice to form one or more patient-derived xenograft (PDX) models. By observing treatment response of these PDX models, determinations can be made about the corresponding patient from which the tumor cells and/or tissue came. Thus, using the tumor dynamics models in the embodiments described herein may allow for improved delivery of precision medicine to patients, where treatment plans are tailored to a patient's particular disease or condition. In this manner, the specific gene profile of the patient's tumor is used to determine or otherwise influence a selected treatment protocol for the patient.

The embodiments described herein recognize that using RNA sequencing data (e.g., mRNA expression levels) for tumor dynamics modeling may improve model accuracy and thus overall accuracy of the computer system specially configured to run the model. Thus, the embodiments described herein provide one or more technical benefits, which may include, for example, without limitation, improving the performance (e.g., accuracy) of a model and/or improving the performance (e.g., accuracy) of a computer system that is specially configured to run the model to perform tumor dynamics modeling.

For example, the embodiments described herein provide a neural ordinary differential equation (neural ODE or NODE) system that receives an input embedding that is a fusion (e.g., concatenation) of two different embeddings—a first embedding generated by a tumor encoder based on low-dimensional observed tumor data and a second embedding generated by a heterogeneous graph encoder based on high-dimensional omics-based data. The first embedding may be a tumor embedding that represents observed tumor data for one or more PDX models over a limited observational window. The second embedding may be graph embedding that is itself a fusion (e.g., concatenation) of three different embeddings—a gene-gene embedding, a drug-gene embedding, and a disease-gene embedding. The gene-gene embedding may be a representation of the interactions between genes as determined using the gene domain of a heterogeneous graph and a graph convolutional network (GCN) system. The heterogeneous graph may include gene nodes that belong to the gene domain, drug nodes that belong to a drug domain, and disease nodes that belong to a disease domain. The drug-gene embedding may be a representation of the genes that are targeted by drugs as determined using the drug nodes and gene nodes of the heterogeneous graph. The disease-gene embedding may be a representation of the genes that are associated with different diseases as determined using the disease nodes and gene nodes of the heterogeneous graph.

The neural ODE system receives both the tumor embedding and the graph embedding as input and processes these embeddings to generate an embedding that is input into a reducer to generate a prediction. The prediction may include, for example, predicted tumor size, a predicted treatment response category, and/or one or more other predicted tumor metrics with respect to a future point in time. The neural ODE system described herein that uses the embeddings generated by both a tumor encoder and heterogeneous graph encoder as described herein may outperform conventional models or neural ODE system that uses only the embedding provided by the tumor encoder.

Thus, the embodiments described herein improve one or more technical fields, such as for example, the technical field of oncology. For example, the embodiments described herein improve the technical field of oncology by providing more accurate—compared to conventional systems—detection, diagnosis, and/or treatment of tumor and/or tumor growth. This example improvement is due to the described embodiments providing a technical solution (e.g., modeling tumor dynamics using RNA sequencing data) to a technical problem (e.g., conventional tumor dynamics modeling techniques are associated with less accurate modeling and lowered ability to handle genomics data).

In some embodiments, the embodiments described herein include an unconventional combination of steps that results in improvements to the technical field of oncology and/or tumor dynamics modeling. For example, the combination of steps associated with the use of RNA sequencing data is associated with predictions of tumor growth over time that more accurately models tumor dynamics.

The methodologies and systems described herein may further be used to generate specific treatment-related outputs for use in managing the administration of a treatment for tumors. The treatment may include, for example, without limitation, at least one of a small molecule inhibitor of SHP2, a neoantigen cancer vaccine, a T-cell therapy, a personalized cancer vaccine, a neoantigen-directed T cell therapy, an immunotherapeutic, or some other type of oncology or tumor treatment. The tumor treatment may include, for example, at least one of alectinib, bevacizumab, glofitamab-gxbm, cobimetinib, vismodegib, obinutuzumab, trastuzumab, hyaluronidase (e.g., hyaluronidase-oysk, hyaluronidase-zzxf, hyaluronidase human, and/or one or more other types of hyaluronidase,), ado-trastuzumab emtansine, mosunetuzumab-axgb, pertuzumab, polatuzumab vedotin-piiq, rituximab, entrectinib, erlotinib, atezolizumab, venetoclax, capecitabine, or vemurafenib. The treatment may include, for example, at least one of clofarabine, cimetidine, thiamine, or arsenic trioxide. The treatment may include, for example, at least one of dextromethorphan, solifencacin, atomoxetine, venlafaxine, or tapentadol.

The tumor or disease may be, for example, a tumor associated with soft tissue sarcoma, pancreatic ductal carcinoma, esophageal cancer, ovarian carcinoma, renal cell carcinoma, breast carcinoma, colorectal cancer, non-small cell lung carcinoma, cutaneous melanoma, and/or some other disease. The origin of the tumor may be, for example, colorectal, soft tissue, liver, ovary, breast, lung, skin, kidney, endometrium, central nervous system, head and/or neck, lymphoma, stomach, pancreas, esophagus, and/or some other organ or tissue.

The predictions generated by of the systems described herein may be used to perform any number of treatment actions. For example, a decision to administer a candidate treatment to a subject may be made based on the prediction. A change may be made to the type of treatment and/or the treatment regimen associated with a given treatment based on the prediction. In some cases, a prediction of a less than desired response to a candidate treatment may indicate that a different treatment and/or a different treatment regimen (e.g., dosage/schedule) is to be administered to the subject. Using the systems and methods described herein may thus improve clinical outcomes for subjects.

Accordingly, a desire exists for methods and systems that improve the accuracy of tumor dynamics modeling and improve upon customizing input data sets for such tumor dynamics models based on both low-dimensional and high-dimensional data for a particular patient. Recognizing and taking into account the importance and utility of a methodology and system that can provide these improvements, the specification describes various embodiments for using deep learning to predict tumor dynamics.

### II. Example System for a Tumor Dynamics Prediction System

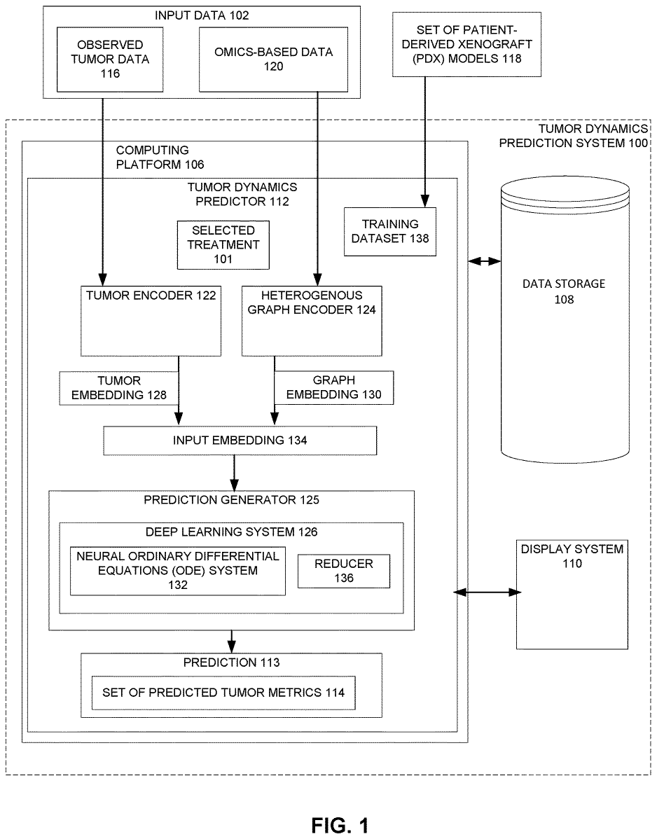

FIG. 1 is a block diagram of tumor dynamics prediction system 100 in accordance with one or more example embodiments. Tumor dynamics prediction system 100 may be used to predict at least one tumor metric that indicates how a tumor will respond to a selected treatment 101. For example, tumor dynamics prediction system 100 may use input data 102 that includes a combination of observed tumor data (e.g., tumor size data) and omics-based data to predict tumor size (e.g., tumor volume) at a future point in time after selected treatment 101 has been administered. Selected treatment 101 may include, for example, without limitation a drug treatment that includes one or more different drugs.

In one or more embodiments, the selected treatment 101 may include, for example, without limitation, at least one of a small molecule inhibitor of SHP2, a neoantigen cancer vaccine, a T-cell therapy, a personalized cancer vaccine, a neoantigen-directed T cell therapy, an immunotherapeutic, or some other type of oncology or tumor treatment. The selected treatment 101 may include, for example, at least one of alectinib, bevacizumab, glofitamab-gxbm, cobimetinib, vismodegib, obinutuzumab, trastuzumab, hyaluronidase (e.g., hyaluronidase-oysk, hyaluronidase-zzxf, hyaluronidase human, and/or one or more other types of hyaluronidase,), ado-trastuzumab emtansine, mosunetuzumab-axgb, pertuzumab, polatuzumab vedotin-piiq, rituximab, entrectinib, erlotinib, atezolizumab, venetoclax, capecitabine, or vemurafenib. The selected treatment 101 may include, for example, at least one of clofarabine, cimetidine, thiamine, or arsenic trioxide. The selected treatment 101 may include, for example, at least one of dextromethorphan, solifencacin, atomoxetine, venlafaxine, or tapentadol.

The tumor or disease being treated by the selected treatment may include, for example, soft tissue sarcoma, pancreatic ductal carcinoma, esophageal cancer, ovarian carcinoma, renal cell carcinoma, breast carcinoma, colorectal cancer, non-small cell lung carcinoma, cutaneous melanoma, and/or some other disease. The origin of the tumor may be, for example, colorectal, soft tissue, liver, ovary, breast, lung, skin, kidney, endometrium, central nervous system, head and/or neck, lymphoma, stomach, pancreas, esophagus, and/or some other organ or tissue.

The tumor dynamics prediction system 100 may be implemented using hardware, software, firmware, or a combination thereof. In one or more embodiments, tumor dynamics prediction system 100 may include a computing platform 106, a data storage 108 (e.g., database, server, storage module, cloud storage, etc.), and a display system 110.

Computing platform 106 may take various forms. In one or more embodiments, computing platform 106 includes a single computer (or computer system) or multiple computers in communication with each other. In other examples, computing platform 106 takes the form of a cloud computing platform, a mobile computing platform (e.g., laptop, a smartphone, a tablet, etc.), another processor-based device (e.g., a workstation or desktop computer) or a wearable computing device (e.g., a smartwatch), and/or the like or a combination thereof. Computing platform 106 may be or may be part of a client device that is a processor-based device including, for example, a workstation, a desktop computer, a laptop computer, a smartphone, a tablet computer, a wearable apparatus, and/or the like.

Data storage 108 and display system 110 are each in communication with computing platform 106. In some examples, data storage 108, display system 110, or both may be considered part of or otherwise integrated with or part of computing platform 106. Thus, in some examples, computing platform 106, data storage 108, and display system 110 may be separate components in communication with each other, but in other examples, some combination of these components may be integrated together.

Tumor dynamics prediction system 100 includes tumor dynamics predictor 112 that may be implemented using software, hardware, and/or firmware. For example, tumor dynamics predictor 112 may be implemented using computing platform 106. Tumor dynamics predictor 112 receives and processes input data 102 to generate a prediction 113. The prediction 113 may include, for example, without limitation, a set predicted tumor metrics 114 that indicate how a tumor will respond to selected treatment 101. Tumor dynamics predictor 112 includes multiple different deep learning systems that work together to generate set of predicted tumor metrics 114.

Input data 102 may include, for example, without limitation, observed tumor data 116. Observed tumor data 116 may be data that is observed with respect to the tumor of the subject who is receiving or is designated to receive selected treatment 101. In other embodiments, observed tumor data 116 may be data that is observed with respect to a set of patient-derived xenograft (PDX) models 118 (or alternatively, a set of subject-derived xenografts).

A PDX model includes one or more animals (e.g., immunodeficient or humanized mice) that have been engrafted with the tumor of a subject. For example, at least a portion of a tumor in a subject may be surgically extracted (e.g., biopsied) and fragmented into tumor cells or tissue that is then transplanted directly or in combination with one or more additives into one or more mice for engraftment. These mice are preclinical mice that have not received selected treatment 101. The tumor cells or tissue may be allowed to grow, and, in some cases, those tumors may be transplanted into one or more other mice for tumor expansion. The expanded tumors may be used for research or further transplanted into one or more mice to form one or more PDX models for tumor research. In some cases, creating the one or more PDX models may include transplanting tumor cells, tumor tissue, or tumor into a tissue or organ different from which it was just previously or originally derived.

Observed tumor data 116 may correspond to the response of set of PDX models 118 to selected treatment 101 over a selected period of time. For example, observed tumor data 116 may include one or more tumor metrics such as, for example, tumor size and/or tumor shape, that are observed over a selected period of time. Tumor size may include, for example, tumor volume, tumor volume, maximum cross-sectional tumor area, maximum tumor length, and/or maximum tumor width, where length and/or width are relative to preselected axes. The selected period of time may be a limited observational window such that the observed tumor data is considered “early” tumor data. The selected period of time may range from 1 hour to about two months after a reference point in time. For example, the selected period of time may be 3 days, 5 days, 7 days, 10 days, 14 days, 18 days, 21 days, 25 days, 28 days, 35 days, 45 days, or 60 days after the reference point in time. The reference point in time may be, for example, a baseline point in time (e.g., the hour, day, or week) of an initial administration of selected treatment 101 or some other point in time associated with a treatment regimen for selected treatment 101. A treatment regimen may include a schedule for selected treatment 101 and/or dosage information for selected treatment 101 with respect to that schedule.

In addition to observed tumor data 116, input data 102 may further include omics-based data 120 that is high-dimensional relative to observed tumor data 116. Observed tumor data 116 may be considered low-dimensional data relative to omics-based data 120 in that the number of features or variables included in observed tumor data 116 is much fewer. High-dimensional data may include a dataset with a large number of features or variables, which may be larger than the number of samples for which data is included in the dataset. As one example, low-dimensional data may include a dataset with 1, 2, 3, 4, 5, 6, 7, 8, 9, or 10 features or variables. High-dimensional data may include hundreds or thousands of features or variables.

Omics-based data 120 may include data that is associated with, for example, without limitation, at least one of genomics data, epigenomics data, transcriptomics data, proteomics data, metabolomics data, metagenomics data, phenomics data, or other data generated based on the tissue and/or cell sample of the subject. Omics-based data 120 may be generated using various methodologies including, for example, without limitation, next generation sequencing (NGS), mass spectrometry, and/or nuclear magnetic resonance. Omics-based data 120 may include heterogeneous data that represents information about the interactions of and/or associations between genes, drugs, and diseases. For example, omics-based data 120 may include gene information derived from RNA sequencing data, drug information identifying the various genes targeted by different drugs, and disease information identifying the various genes associated with different diseases.

As discussed above, tumor dynamics predictor 112 uses observed tumor data 116 and omics-based data 120 to generate prediction 113. Prediction 113 may include a set of predicted tumor metrics 114 with respect to one or more future points in time. Set of predicted tumor metrics 114 may include, for example, a predicted tumor size (e.g., tumor volume) at a future point in time. The future point in time may be, for example, without limitation, 7 days, 14 days, 21 days, 1 month, 2 months, 3 months, 4 months, 5 months, 6 months, 9 months, 12 months, 15 months, 18 months, 21 months, 24 months, or some other period of time after the reference point in time (e.g., time of initial treatment administration).

Tumor dynamics predictor 112 may include tumor encoder 122, heterogeneous graph encoder 124, and prediction generator 125. Prediction generator 125 may include, for example, deep learning system 126. Tumor encoder 122 receives and processes observed tumor data 116 to generate tumor embedding 128. Tumor embedding 128 may be a representation of observed tumor data 116 that may be used as input for deep learning system 126. In one or more embodiment, tumor encoder 122 may include a recurrent neural network (RNN).

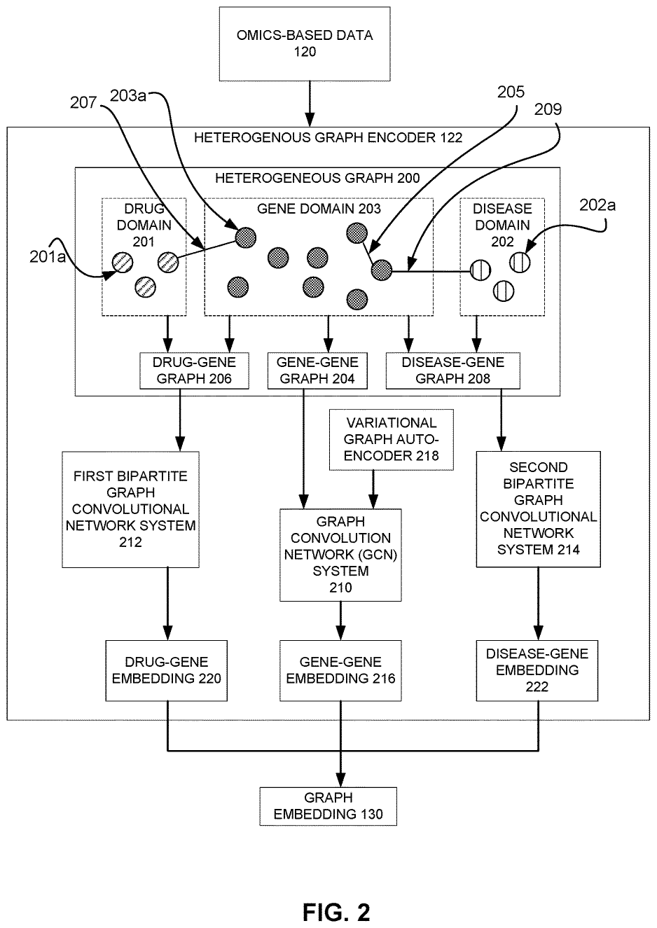

Heterogeneous graph encoder 124 receives and processes omics-based data 120 to generate graph embedding 130 that may be used as input for deep learning system 126. The process of mapping omics-based data 120 to graph embedding 130 is described in greater detail with respect to FIG. 2 below.

Continuing with reference to FIG. 1, deep learning system 126 may receive and process both tumor embedding 128 and graph embedding 130 to generate prediction 113. Prediction 113 may include set of predicted tumor metrics 114. In one or more embodiments, deep learning system 126 includes neural ordinary differential equation (ODE) system 132. Neural ODE system 132 may simulate time evolution of the tumor from the initial time point to one or more future time points using one or more differential equations. Neural ODE system 132 may receive input embedding 134 that includes tumor embedding 128 and graph embedding 130. In some cases, tumor embedding 128 and graph embedding 130 are concatenated together to form input embedding 134. For example, input embedding 134, 8, may be formed by concatenating graph embedding 130, 81, with tumor embedding 128, 82:

$$β = \vert β1 \vert \vert β2 \vert . \qquad ( 1 )$$

Neural ODE system 132 may be used to simulate the time evolution state of y(t):

$$\frac{dy ( t )}{dt} = {f}_{θ} ( y ( t ) , β ) , t ∈ [ 0 , T ] , \qquad ( 2 )$$

where 0 denotes the reference point in time (e.g., start time of PDX experimentation, initial treatment, baseline point in time, etc.), T denotes the end of the prediction time (e.g., the future point in time), f_{θ} is a neural network parameterized by a set of weights θ to be learned across a training dataset 138. The input embedding 134, β, provides the initial condition for the neural ODE system 132 that is specific to the set of PDX models 118 (e.g., single PDX model) of interest. Training dataset 138 may include a PDX dataset that includes data for numerous PDX models covering different types of treatments and diseases. In one or more embodiments, training dataset 136 includes a PDX dataset formed from the data detailed in Gao, H. et al., “High-throughput screening using patient-derived tumor xenografts to predict clinical trial drug response,” Nat Med 21, 1318-1325 (2015), https://doi.org/10.1038/nm.3954, which is incorporated by reference herein in its entirety. Training dataset 138 may include omics-based data (e.g., RNA sequence data), data about tumor size over time, treatment response data, and/or other information.

In one or more embodiments, prediction generator 125 includes a reducer 136 to reduce the dimensions of the output of neural ODE system 132. Reducer 136 may include, for example, a two-layer multi-layer perceptron (MLP) that can be used to reconstruct set of predicted tumor metrics 114—e.g., predict tumor size-based on y (t).

In some embodiments, prediction 113 generated by prediction generator 125 includes a treatment response classification (or treatment response category). The predicted tumor size may be categorized according to Response Evaluation Criteria in Solid Tumors (RECIST) or modified RECIST categories used in clinics for evaluating tumor response to a treatment and treatment decision making. These categories capture a combination of speed, strength, and durability of response into a single value, that is eventually categorized as Complete Response (CR), Partial Response (PR), Stable Disease (SD), Progressive Disease (PD), which helps clinicians in evaluating treatment efficacy and decision making, specifically in clinical trials.

In one or more embodiments, set of predicted tumor metrics 114 may include a predicted tumor size at time t. The difference between the predicted tumor size, Volume(t), and the tumor size at the reference point in time, Volume_{ref}, divided by the tumor size at the reference point in time, Volume_{ref}, may be used to provide a predicted time-dependent tumor response, ΔV(t). A negative percentage value for ΔV(t) indicates tumor shrinkage. This response may be classified based on one or more selected thresholds.

The time-dependent tumor response may be used to define the modified Response Evaluation Criteria in Solid Tumors (mRECIST) categories. For a given dataset, the response of a tumor to a selected treatment 101 may be assigned to a category selected from the following categories: a complete response, a partial response, a stable disease, a progressive disease. The category (or classification) may be assigned by identifying the “best response” over the dataset. The best response may be defined as the minimum value of ΔV(t) for t≥10 days. A best response that is less than −95% indicates a complete response to treatment. A best response that is greater than −95% but less than −50% indicates a partial response to treatment. A best response that is greater than −50% but less than 35% indicates stable disease. A best response that is greater 35% indicates progressive disease.

FIG. 2 is a block diagram of heterogeneous graph encoder 124 from FIG. 1 described in greater detail in accordance with one or more embodiments. As discussed above with respect to FIG. 1, heterogeneous graph encoder 124 receives and processes omics-based data 120 to generate graph embedding 130.

Omics-based data 120 may be a representation of biological and pharmacological knowledge compiled from various sources. Generally, omics-based data 120 may provide information about the associations between different genes, information about the genes that are targeted by different drugs and/or the genes that interact with different drugs, and information about the genes and/or gene variants associated with diseases.

In one or more embodiments, omics-based data 120 includes heterogeneous graph 200. In some embodiments, tumor dynamics predictor 112 of FIG. 1 uses omics-based data 120 to form or build heterogeneous graph 200 and send heterogeneous graph 200 as input into heterogeneous graph encoder 124. In other embodiments, heterogeneous graph encoder 124 uses omics-based data 120 to form or build heterogeneous graph 200.

Heterogeneous graph 200 includes nodes that fall in three domains: the drug domain 201, the disease domain 202, and the gene (or RNA) domain 203. An edge between two nodes in the heterogeneous graph 200 indicates an association between the two nodes. The three different domains may form different subgraphs within heterogeneous graph 200. Drug node 201a is an example of a node in the gene domain 203. Disease node 202a is an example of a node in the disease domain 202. Gene node 203a is an example of a node in the gene domain 203.

Heterogeneous graph 200 may be, for example, an undirected graph, G(V, E), with three different sets of nodes including, for example, a set of drug nodes (V^{A}) corresponding to drug domain 201, a set of disease nodes (V^{B}) corresponding to disease domain 202, and a set of gene nodes (V^{C}) corresponding to gene (or RNA sequence) domain 203. The initial features of these three sets of nodes may be represented by the feature vectors or matrices X_{Va}, X_{Vb}, and X_{Vc}, respectively. The edges of the undirected graph include three different types of edges, each type representing a different type of association. For example, the edges include a set of interdomain edges representing drug-gene associations (E^{AC}), a set of interdomain edges representing disease-gene associations (E^{BC}), and a set of intradomain edges representing gene-gene associations (E^{CC}). Accordingly, heterogeneous graph 200 may be considered a fusion of three different graphs formed by the various nodes and edges described above, the three graphs including a gene-gene graph 204 (e.g., edge 205 may be an example of an edge in the gene-gene graph 204), a drug-gene graph 206 (e.g., edge 207 may be an example of an edge in the drug-gene graph 206), and a disease-gene graph 208 (e.g., edge 209 may be an example of an edge in the disease-gene graph 208).

Gene-gene graph 204 may be, for example, without limitation, a gene network system that includes one or more functional gene networks comprised of gene nodes and intradomain edges between the gene nodes. For example, gene-gene graph 204 may include a set of tissue-specific functional gene networks (FGNs) that provide gene-gene associations based on gene expression across multiple tissues. In one or more examples, a node of the functional gene network (FGN) of the gene-gene graph 204 represents a gene, an edge between two nodes represents an association between two genes, and the edge weight associated with an edge indicates the co-functional probability that those two genes participate in the same biological pathway.

In one or more embodiments, gene-gene graph 204 may include or may be built from multiple functional gene networks obtained from TissueNexus, a database of tissue-specific functional gene networks (FGNs) for at least 49 human tissues and/or cell lines, available at https://www.diseaselinks.com/TissueNexus/, and described in Cui-Xiang Lin et al., “TissueNexus: a database of human tissue functional gene networks built with a large compendium of curated RNA-seq data,” Nucleic Acids Research, Volume 50, Issue D1, 7 Jan. 2022, pages D710-D718, https://doi.org/10.1093/nar/gkab1133, which is incorporated by reference herein in its entirety.

Drug-gene graph 206 may be, for example, without limitation, a graph that represents the interactions between drugs and genes. In drug-gene graph 206, the nodes may include drug nodes and gene nodes, and the edges may be interdomain edges formed between the drug nodes and gene nodes. The gene nodes may represent genes and/or gene variants. For example, an edge between a drug node and a gene node may indicate an interaction or association between the corresponding drug and corresponding gene, respectively. For example, the drug may target or otherwise interact with that gene. In one or more embodiments, drug-gene graph 206 is obtained from or otherwise built using the data about drug-gene interactions provided by DGIdb, a database that aggregates genes or gene products, drugs and drug-gene interaction records from multiple disparate sources via expert curation and text mining, available at https://www.dgidb.org/, and described in Matthew Cannon et al., “DGIdb 5.0: rebuilding the drug-gene interaction database for precision medicine and drug discovery platforms,” Nucleic Acids Research, Volume 52, Issue D1, 5 Jan. 2024, pages D1227-D1235, https://doi.org/10.1093/nar/gkad1040, which is incorporated by reference herein in its entirety.

Disease-gene graph 208 may be, for example, without limitation, a graph that represents the association between genes and diseases. In disease-gene graph 208, the nodes include disease nodes and gene nodes with interdomain edges formed between the disease nodes and gene nodes. The gene nodes may represent genes and/or gene variants. An edge between a disease node and a gene node (representing a gene or gene variant) indicates an association between the corresponding disease and the corresponding gene or gene variant, respectively. In one or more embodiments, disease-gene graph 208 is obtained from or otherwise built using the data about disease-gene associations provided by DISGENET, available at https://disgenet.com/, and described in Janet Piñero et al., “The DisGeNET knowledge platform for disease genomics: 2019 update, Nucleic Acids Research,” Volume 48, Issue D1, 8 Jan. 2020, pages D845-D855, https://doi.org/10.1093/nar/gkz1021, which is incorporated by reference herein in its entirety. Version 24.3 of DISGENET is a compendium of nearly 1,943,710 gene-disease associations (GDAs), between 26,484 genes and 39,922 diseases and traits; 1,237,183 variant-disease associations (VDAs), between 723,970 variants and 16,938 diseases and traits, and over 27,494,442 M disease-disease associations.

Heterogeneous graph encoder 124 may include graph convolutional network (GCN) system 210, first bipartite graph convolutional network (GCN) system 212, and second bipartite graph convolutional network (GCN) system 214. GCN system 210 may include one or more multi-layer graph convolutional neural networks that are applied to the gene domain of heterogeneous graph 200 to model the interactions between genes and generate gene-gene embedding 216 that represents those interactions. For example, GCN system 210 may be applied to gene-gene graph 204 to generate gene-gene embedding 216.

The model parameters of GCN system 210 and the features of the gene nodes may be initialized using a pretrained variational graph auto-encoder (VGAE) 218. One example of an implementation for a loss function that may be used to pretrain variational graph auto-encoder 218 is described in Thomas N Kipf and Max Welling, “Variational graph auto-encoders,” arXiv preprint arXiv: 1611.07308, which is incorporated by reference herein in its entirety. Variational graph auto-encoder 218 may be pretrained or fine-tuned based on the RNA sequence data (e.g., mRNA expression data) in training dataset 138.

In one or more embodiments, three different losses may be used to pretrain the VGAE 218 that includes two GCN layers. The three different losses may include node reconstruction, edge reconstruction, and Küllback-Leibler divergence (KL-divergence). The node reconstruction loss may be the mean squared error (MSE) of genes (nodes of the input graph) and reconstructed genes (decoder output). The edge reconstruction loss may be the cross entropy of positive and negative edges in the graph. The KL-divergence may be used as a regularization term in the loss function to assure continuity and completeness in the latent space.

In one or more embodiments, first bipartite graph convolutional network (GCN) system 212 may include a single bipartite graph attention convolution layer that propagates messages from drugs to target genes. For example, drug-gene graph 206 may be input into first bipartite GCN system 212 to generate drug-gene embedding 220. Applying the single bipartite graph attention convolution layer to drug-gene graph 206 may be considered to be projecting information from a macro level (e.g., drug domain 201) to the micro level (e.g., gene domain 203).

In one or more embodiments, second bipartite graph convolutional network (GCN) system 214 may include a single bipartite graph attention convolution layer that is used to project the gene domain 203 to the disease domain 202 to generate disease-gene embedding 222. For example, disease-gene graph 208 may be input into second bipartite GCN system 214 to generate disease-gene embedding 222. The non-linear graph information captured by the gene nodes may be used to update the hidden embeddings of the disease nodes. Applying the single bipartite graph attention convolution layer to disease-gene graph 208 may be considered an attentional pooling of the disease-gene graph 208.

Heterogeneous graph encoder 124 may fuse gene-gene embedding 216, drug-gene embedding 220, and disease-gene embedding 222 to form graph embedding 130. For example, gene-gene embedding 216, drug-gene embedding 220, and disease-gene embedding 222 may be concatenated to form graph embedding 130 that is used as input for neural ODE system 132.

Tumor dynamics prediction system 100 described with respect to FIG. 1 and FIG. 2 provides an improvement to the technical field of tumor dynamics modeling because tumor dynamics prediction system 100 provides an end-to-end learning framework that integrates high-dimensional data (e.g., omics-based data 120) with low-dimensional data (e.g., observed tumor data 116).

In some embodiments, the improvement to the technical field of dynamic modeling is a result of the combination of tumor encoder 122, the heterogenous graph encoder 124, which allows for feature extraction from raw graph-structured data, the neural ODE system 132, which achieves better performance in both data fitting and prediction settings than conventional approaches. Using both embeddings from the tumor encoder 122 and the heterogenous graph encoder 124 allows representations of both high-dimensional data and low-dimensional data to be fed into the neural ODE system 132, to thereby enhance the quality of latent space representation and improve model predictive performance, especially where observed tumor data 116 is over a limited observation window.

### III. Example Methodologies

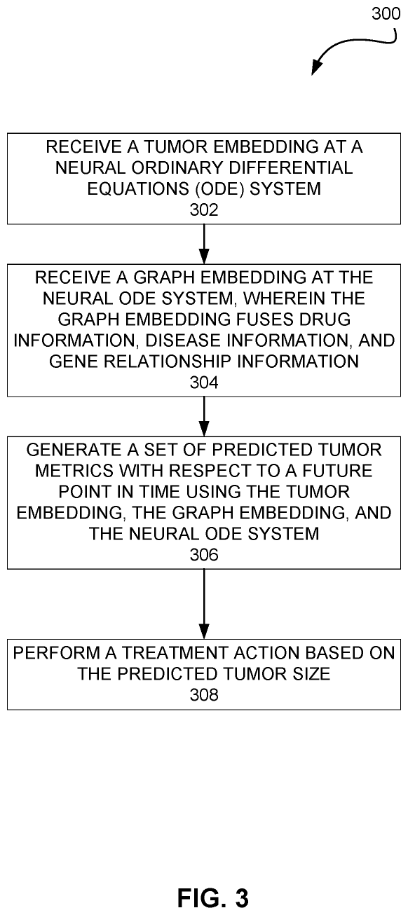

FIG. 3 is a flowchart illustrating an embodiment of a process for predicting tumor dynamics in accordance with one or more embodiments. In one or more embodiments, process 300 may be implemented using tumor dynamics prediction system 100 described in FIG. 1 and FIG. 2. Accordingly, the process 300 in FIG. 3 is described with continuing reference to FIG. 1 and FIG. 2 and such that the description of process 300 may apply to tumor dynamics prediction system 100 in FIG. 1 and FIG. 2. One or more steps that are not expressly illustrated in FIG. 3 may be included before, after, in between, or as part of the steps of process 300. In some embodiments, process 300 may begin with step 302.

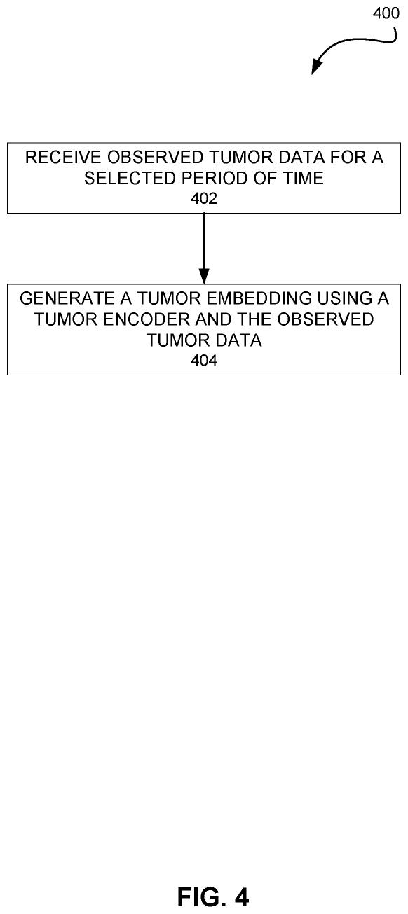

Step 302 includes receiving a tumor embedding at a neural ordinary differential equations (ODE) system. The neural ODE system may be, for example, neural ODE system 132 in FIG. 1. The tumor embedding, which may be, for example, tumor embedding 128 in FIG. 1, may be a representation of observed tumor data over a selected period of time after a reference point in time. One example of an implementation for generating tumor embedding is described with respect to FIG. 4 below.

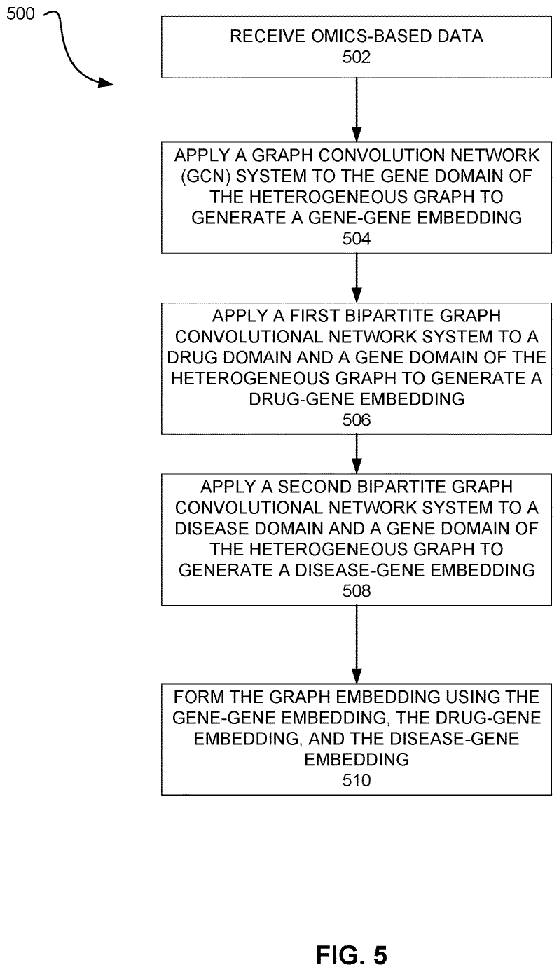

Step 304 includes receiving a graph embedding at the neural ODE system, wherein the graph embedding fuses drug information, disease information, and gene relationship information. The graph embedding, which may be, for example, graph embedding 130 in FIG. 1, may be a representation of the interactions between genes, drugs, and diseases that provide a baseline state for analysis. The graph embedding may represent, for example, gene-gene associations, drug-gene associations, and disease-gene associations. One example of an implementation for generating graph embedding is described with respect to FIG. 5 below.

Step 306 includes generating a set of predicted tumor metrics with respect to a future point in time using the tumor embedding, the graph embedding, and the neural ODE system. The set of predicted tumor metrics, which may be, for example, set of predicted tumor metrics 114 in FIG. 1, may include predicted tumor size at the future point in time. In some cases, set of predicted tumor metrics 114 may further include a predicted growth or shrinkage rate over a period of time between the reference point and the future point in time.

Step 308 includes performing a treatment action based on the predicted tumor size. Step 308 may include, for example, administering a candidate tumor treatment to a subject in response to the predicted tumor size being less than a selected threshold. The candidate tumor treatment may be the same treatment for which the observed tumor data was provided. The candidate tumor treatment may include a single drug or multiple drugs in combination. In some embodiments, step 308 includes administering a tumor treatment other than the candidate tumor treatment to the subject in response to the predicted tumor size being greater than the selected threshold.

In one or more embodiments, step 308 may include, for example, adjusting a treatment regimen for the candidate tumor treatment based on the predicted tumor size. For example, adjusting the treatment regimen may include changing a treatment schedule (e.g., adjusting a dosage frequency or treatment interval), changing a treatment dosage, or both. In some cases, step 308 may include maintaining a current treatment or current treatment regimen being administered to a subject based on the predicted tumor size.

The treatment associated with step 308 may include, for example, without limitation, at least one of a small molecule inhibitor of SHP2, a neoantigen cancer vaccine, a T-cell therapy, a personalized cancer vaccine, a neoantigen-directed T cell therapy, an immunotherapeutic, or some other type of oncology or tumor treatment. The treatment may include, for example, at least one of alectinib, bevacizumab, glofitamab-gxbm, cobimetinib, vismodegib, obinutuzumab, trastuzumab, hyaluronidase (e.g., hyaluronidase-oysk, hyaluronidase-zzxf, hyaluronidase human, and/or one or more other types of hyaluronidase,), ado-trastuzumab emtansine, mosunetuzumab-axgb, pertuzumab, polatuzumab vedotin-piiq, rituximab, entrectinib, erlotinib, atezolizumab, venetoclax, capecitabine, or vemurafenib. The treatment may include, for example, at least one of clofarabine, cimetidine, thiamine, or arsenic trioxide. The treatment may include, for example, at least one of dextromethorphan, solifencacin, atomoxetine, venlafaxine, or tapentadol.

The tumor or disease being treated by the treatment may include, for example, soft tissue sarcoma, pancreatic ductal carcinoma, esophageal cancer, ovarian carcinoma, renal cell carcinoma, breast carcinoma, colorectal cancer, non-small cell lung carcinoma, cutaneous melanoma, and/or some other disease. The origin of the tumor may be, for example, colorectal, soft tissue, liver, ovary, breast, lung, skin, kidney, endometrium, central nervous system, head and/or neck, lymphoma, stomach, pancreas, esophagus, and/or some other organ or tissue.

In some embodiments, the outputs of step 306 and/or step 308, and thereby process 300, are an improvement in the technical field of dynamic tumor modeling because the process 300 provides end-to-end learning that integrates high-dimensional omics-based data (e.g., RNA sequence data) with low-dimensional observed tumor data (e.g., tumor size or tumor volume data). The process 300 enables more accurate prediction of treatment response and earlier prediction with a more limited observational window than possible with some currently available conventional approaches. Further, using a neural ODE system that uses both a tumor embedding and graph embedding as input provides improved performance in both data fitting and prediction settings than conventional approaches. For example, the integration of a graph embedding provided by a heterogenous graph encoder with the neural ODE system may enhance the quality of latent space representation and improve the model predictive performance, especially with limited observation data.

In one or more embodiments, the steps in the process 300 are a new combination of steps that results in a technical improvement in the field of dynamic tumor modeling. In one or more embodiments, the combination of steps in the process 300 is a non-conventional and non-generic arrangement, which results in end-to-end learning that integrates high-dimensional RNA-seq data.

FIG. 4 is a flowchart illustrating a process for generating a tumor embedding in accordance with one or more embodiments. In one or more embodiments, process 400 may be implemented using tumor dynamics prediction system 100 described in FIG. 1 and FIG. 2. Accordingly, the process 400 in FIG. 4 is described with continuing reference to FIG. 1 and FIG. 2 and such that the description of process 400 may apply to tumor dynamics prediction system 100 in FIG. 1 and FIG. 2. Further, the process 400 is an example of one implementation of a process for generating the tumor embedding received in step 302 in FIG. 3. One or more steps that are not expressly illustrated in FIG. 4 may be included before, after, in between, or as part of the steps of process 400. In some embodiments, process 400 may begin with step 402.

Step 402 includes receiving observed tumor data for a selected period of time. The observed tumor data may be, for example, observed tumor data 116 in FIG. 1. The observed tumor data may be data that is observed with respect to the tumor of the subject who is receiving or is designated to receive a selected treatment. The selected treatment may be, for example, a candidate treatment that includes a single drug or multiple drugs in combination. The observed tumor data may be data observed with respect to the response of a set of patient-derived xenograft (PDX) models to the selected treatment over a selected period of time. For example, the observed tumor data may include one or more tumor metrics such as, for example, tumor size and/or tumor shape, which are observed over a selected period of time. Tumor size may include, for example, tumor volume, tumor volume, maximum cross-sectional tumor area, maximum tumor length, and/or maximum tumor width, where length and/or width are relative to preselected axes.

The selected period of time may be a limited observational window such that the observed tumor data is considered “early” tumor data. The selected period of time may range from 1 hour to about two months after a reference point in time. For example, the selected period of time may be 3 days, 5 days, 7 days, 10 days, 14 days, 18 days, 21 days, 25 days, 28 days, 35 days, 45 days, or 60 days after the reference point in time. The reference point in time may be, for example, a baseline point in time (e.g., the hour, day, or week) of an initial administration of the selected treatment or some other point in time associated with a treatment regimen for selected treatment. A treatment regimen may include a schedule for the selected treatment and/or dosage information for the selected treatment with respect to that schedule.

Step 404 includes generating a tumor embedding using a tumor encoder and the observed tumor data. The tumor encoder may include, for example, a recurrent neural network (RNN). For example, step 404 may include applying the recurrent neural network to the observed tumor data to generate the tumor embedding.

FIG. 5 is a flowchart illustrating a process for generating a graph embedding in accordance with one or more embodiments. In one or more embodiments, process 400 may be implemented using tumor dynamics prediction system 100 described in FIG. 1 and FIG. 2. Accordingly, the process 500 in FIG. 5 is described with continuing reference to FIG. 1 and FIG. 2 and such that the description of process 500 may apply to tumor dynamics prediction system 100 in FIG. 1 and FIG. 2. Further, the process 500 is an example of one implementation of a process for generating the graph embedding received in step 304 in FIG. 3. One or more steps that are not expressly illustrated in FIG. 5 may be included before, after, in between, or as part of the steps of process 500. In some embodiments, process 500 may begin with step 502

Step 502 includes receiving omics-based data, which may be, for example, omics-based data 120 in FIG. 1. In some embodiments, the omics-based data takes the form of a heterogeneous graph, such as, for example, heterogenous graph 200 in FIG. 2. In other embodiments, the heterogeneous graph may be constructed using the omics-based data.

For example, optionally, where omics-based data is not represented in graph form, the process 500 may include step 504, which includes building a heterogeneous graph using the omics-based data. Whether the heterogeneous graph is built at step 504 or received in step 502, the heterogeneous graph includes nodes that belong to three different domains. These three different domains include the drug domain (e.g., drug domain 201 in FIG. 2), the disease domain (e.g., disease domain 202 in FIG. 2), and the gene domain (e.g., gene domain 203 in FIG. 2).

The heterogeneous graph may be, for example, an undirected graph, G(V, E), with three different sets of nodes including, for example, a set of drug nodes (V^{A}) corresponding to the drug domain, a set of disease nodes (V^{B}) corresponding to the disease domain, and a set of gene nodes (V^{C}) corresponding to the gene (or RNA sequence) domain. The initial features of these three sets of nodes may be represented by the feature vectors or matrices X_{Va}, X_{Vb}, and X_{Vc}, respectively. The edges of the undirected graph include three different types of edges, each type representing a different type of association. For example, the edges include a set of interdomain edges representing drug-gene associations (E^{AC}), a set of interdomain edges representing disease-gene associations (E^{BC}), and a set of intradomain edges representing gene-gene associations (E^{CC}). Accordingly, the heterogeneous graph may be considered a fusion of three different graphs (or subgraphs) formed by the various nodes and edges described above, the three graphs (or subgraphs) including a gene-gene graph (e.g., gene-gene graph 204 in FIG. 2), a drug-gene graph (e.g., drug-gene graph 206 in FIG. 2), and a disease-gene graph (e.g., disease-gene graph 208 in FIG. 2).

Step 504 may include applying a graph convolution network (GCN) system to the gene domain of the heterogeneous graph to generate a gene-gene embedding. The GCN system, which may be, for example, GCN system 210 in FIG. 2, may include one or more multi-layer graph convolutional neural networks that are applied to the gene domain of heterogeneous graph 200 to model the interactions between genes. The GCN system may generate gene-gene embedding (e.g., gene-gene embedding 216) that represents those gene-gene interactions. In one or more embodiments, applying the GCN system to the gene domain in step 504 performs convolutional operations over the genes that are typically co-expressed in a tissue of interest (e.g., neighboring genes) to output the gene-gene embedding.

The model parameters of the GCN system and the feature vectors or matrices of the gene nodes may be initialized using a pretrained variational graph auto-encoder (e.g., VGAE 218 in FIG. 2). The variational graph auto-encoder may be pretrained based on the RNA sequence data (e.g., mRNA expression data) in a training dataset comprised of data for a plurality of patient-derived xenograft (PDX) models. In one or more embodiments, the training dataset includes over 1000 PDX models, each of which is characterized by baseline mRNA expression levels prior to treatment. The training dataset may include data covering over 60 distinct treatments across 5 or more diseases, with tumor metric measurements (e.g., tumor volume measurements) being taken every two or three days. Using the pretrained variational graph auto-encoder to initialize the GCN system helps reduce computing resources and improve efficiency because otherwise, the gene-gene graph (or subgraph) of the heterogeneous graph 200 might be too large and/or unwieldly for prediction-related tasks.

Step 506 includes applying a first bipartite graph convolutional network system to a drug domain and a gene domain of the heterogeneous graph to generate a drug-gene embedding. The first bipartite graph convolutional network system, which may be first bipartite graph convolutional network system 212 in FIG. 2, includes a bipartite graph attention convolution layer. The first bipartite graph convolutional network system may be used to project the drug domain to the gene domain (e.g., pass messages from the drug nodes to the gene nodes) to generate drug-gene embedding 220.

A bipartite graph may be represented as BG(U, V, E), defined as a graph G(U ∪ E), where U and V represent two sets of nodes corresponding to two respective domains. All edges in the bipartite graph connect nodes from U to V. Each node may be represented by a feature vector or matrix.

In step 506, the drug domain may be the V nodes and the gene domain may be the U nodes. To message pass (MP) from the drug domain, V, to the gene domain, U, the bipartite graph convolution (bg) may be represented as follows:

$$b {g}_{E} ( {u}_{i} ) = ρ ( agg ( \{ {W}_{{u}_{i} , {v}_{j}} , {\vec{x}}_{{v}_{j}} \vert {v}_{j} ∈ {N}_{{u}_{i}}^{E} \} ) ) , \qquad ( 3 )$$

where

$${N}_{{u}_{i}}^{ε}$$

represents the neighborhood of node uᵢ,
which is connected by E in BG(U, V, E)

$$( {N}_{{u}_{i}}^{ε} ⊂ V ) ,$$

where Wᵤ_{ᵢ}_{,v}_{ⱼ} ∈ [symbol] is a feature weighting kernel transforming N-dimensional features to M-dimensional features,
where i and j are the i-th and j-th node in U and V, respectively,
where agg is a permutation-invariant aggregation operation, and
where the ρ operator can be a non-linear activation function.

For agg, element-wise mean-pooling is used. For the ρ operator, ReLU (rectified linear unit) activation is used. Further, the bipartite graph convolution layers utilize the graph attention network as the backbone on the node features to result in the bipartite graph attention convolution layer (bga).

Because the attention mechanism considers the features of nodes in two domains, a learnable matrix for the features is defined as:

$${W}^{u} ∈ \text{?} , for the X features of the U domain , and \qquad ( 4 )$$

$${W}^{v} ∈ \text{?} , for the X features of the V domain , \qquad ( 5 )$$

where P, Q, and S are nodes of the message passing graphs in the respective domains.

The bipartite graph attention convolution layer (bga) may then be defined as:

$$b g {a}_{E} ( {u}_{i} ) = R e L U ( {\sum }_{{v}_{j} ∈ {N}_{{u}_{i}}^{E}} {α}_{{u}_{i} , {v}_{j}} {W}^{v} {\vec{x}}_{{v}_{j}} ) \qquad ( 6 )$$

The attention weight coefficients can be expressed as:

$${α}_{{u}_{i} , {v}_{j}} = \frac{exp ( ρ ( {\vec{α}}^{T} [ {W}^{u} {\vec{x}}_{{u}_{j}} \vert \vert {W}^{v} {\vec{x}}_{{v}_{j}} ] ) )}{{\sum }_{{v}_{k} ∈ {N}_{{u}_{i}}^{E}} exp ( ρ ( {\vec{α}}^{T} [ {W}^{u} {\vec{x}}_{{u}_{j}} \vert \vert {W}^{v} {\vec{x}}_{{v}_{j}} ] ) )} \qquad ( 7 )$$

where T and ∥ represent the matrix transposition and concatenation operations, respectively.

In step 506, to formulate the message-passing step from the drug to gene domain, the hidden embeddings of the node

$${v}_{i}^{c}$$

$$as$$

$${h}_{{v}_{i}^{c}}^{( k )} ,$$

where k is the step index and when

$$k = 0 ,$$

$${h}_{{v}_{i}^{c}}^{( k )} = {x}_{{v}_{i}^{c}} .$$

The term

$${h}_{{v}_{i}^{c}}^{( k )} ,$$

for the hidden embeddings (feature representations) of the gene nodes, may be computed as follows:

$${MP}_{{V}^{A} \to {V}^{C}}^{( k )} : {h}_{{v}_{i}^{c}}^{( k )} = b g {a}_{{E}^{A C}} ( {v}_{i}^{c} ) + {h}_{{v}_{i}^{c}}^{( k - 1 )} . \qquad ( 8 )$$

Step 508 may include applying a second bipartite graph convolutional network system to a disease domain and a gene domain of the heterogeneous graph to generate a disease-gene embedding. The second bipartite graph convolutional network system, which may be second bipartite graph convolutional network system 214 in FIG. 2, includes a bipartite graph attention convolution layer. The second bipartite graph convolutional network system may be used to project the gene domain to the disease domain (e.g., pass messages from the gene nodes to the disease nodes) to generate disease-gene embedding 222.

Step 508 may be performed in a manner similar to step 506 described above. The updated hidden embeddings (feature representations) of the disease nodes may be computed as follows:

$${MP}_{{V}^{C} \to {V}^{B}}^{( k )} : {h}_{{v}_{i}^{b}}^{( k )} = b g {a}_{{E}^{CB}} ( {v}_{i}^{b} ) + {h}_{{v}_{i}^{b}}^{( k - 1 )} . \qquad ( 9 )$$

Step 510 may include forming the graph embedding using the gene-gene embedding, the drug-gene embedding, and the disease-gene embedding. For example, step 510 may be performed by concatenating the gene-gene embedding, the drug-gene embedding, and the disease-gene embedding to form the graph embedding, which may be, for example, graph embedding 130 in FIG. 1.

In one or more embodiments, the steps in the process 500 are a new combination of steps that results in a technical improvement in the field of dynamic tumor modeling. In one or more embodiments, the combination of steps in the process 500 is a non-conventional and non-generic arrangement, which results in end-to-end learning that integrates high-dimensional RNA-seq data. In one or more embodiments, the new combination of steps provides better performance in both data fitting and prediction settings than conventional approaches; enhancements to the quality of latent space representation; and improves the model predictive performance, especially with limited observation data. The process used for encoding the heterogeneous graph allows for rich feature extraction from raw graph-structure data.

Further, in one or more embodiments, the process 300 in FIG. 3, the process 400 in FIG. 4, and/or the process 500 in FIG. 5 include a new and unconventional combination of steps. The new and unconventional combination of steps in the process 300 in FIG. 3, the process 400 in FIG. 4, and/or the process 500 in FIG. 5 allows tumor dynamics prediction system 100 described with respect to FIG. 1 and FIG. 2 to achieve better performance in both data fitting and prediction settings than conventional approaches, provide enhancements to the quality of latent space representation, and improve the model predictive performance, especially with limited observation data.

### IV. Example Results

An example study was conducted in accordance with the embodiments described above with respect to a tumor dynamics prediction system implemented in a manner such as tumor dynamics prediction system 100 in FIG. 1 and FIG. 2 and the processes 300, 400, and 500 described with respect to FIGS. 3, 4, and 5, respectively. The primary dataset used for this study was obtained from a large-scale pre-clinical study conducted in PDX mice models. This dataset, which may be an example of one implementation for training dataset 138 in FIG. 1, included over 1000 PDX models, each characterized by their baseline mRNA expression levels prior to treatment. The dataset covered 62 distinct treatments across six different diseases, with tumor volume measurements taken every 2-3 days. For this study, based on the availability of RNA-seq data, data from 191 unique tumors and 59 different treatments was included, resulting in a comprehensive dataset of 3470 PDX experiments (consisting of various tumor and treatment combinations) spanning 5 tumor types.

The study was performed to evaluate the capability of the tumor dynamics prediction system 100 (e.g., including the neural ODE system) to capture longitudinal tumor volume data in comparison to conventional models that predict tumor growth inhibition metrics (e.g., TGI models). All tumor volume data for all PDX models in the dataset were used. The longitudinal tumor volume data for the dataset was encoded into a latent space using a tumor encoder (also referred to as a tumor volume encoder). The latent space was then used as part of the initial condition to solve an ODE system using a neural ODE system (e.g., neural ODE system 132). For example, for each PDX model of interest, observed tumor data (e.g., tumor volume over different selected periods of time) was input into a tumor encoder (e.g., a recurrent neural network) to generate a tumor embedding in a latent space. The selected periods of time (observational windows) were 7 days, 14 days, 21 days, and 28 days, to simulate real-world scenarios where early observations are used to forecast future tumor volume dynamics.

The tumor dynamics prediction system outperformed the conventional TGI models as shown below in Table 1 by the R2 parameter and Spearman correlation parameter.

TABLE 1

| | Conventional TGI | Tumor Dynamics |
|---|---|---|
| | Model | Prediction System |
| R2 | 0.71 | 0.96 |
| Spearman correlation | 0.86 | 0.96 |

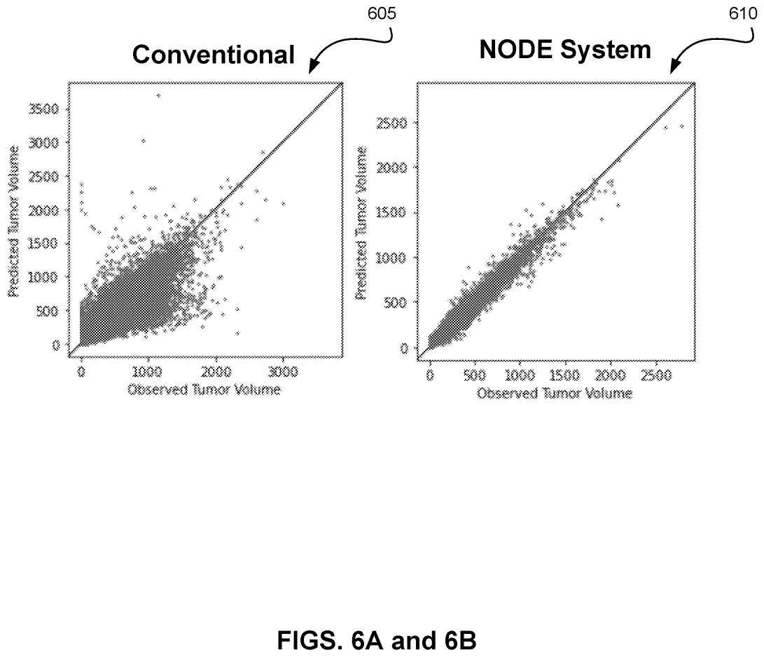

FIGS. 6A and 6B together provide a graphical comparison of tumor volume trace fitting using conventional technology to tumor volume trace fitting using the tumor dynamics prediction system described herein in accordance with one or more example embodiments. The predicted tumor volumes generated using conventional technology (e.g., a TGI model) are plotted against the actual tumor volumes in plot 605 of FIG. 6A. The predicted tumor volumes generated using the tumor dynamics prediction system (e.g., tumor dynamics prediction system 100 described in FIG. 1 and FIG. 2 along with the processes 300, 400, and 500 described in FIGS. 3, 4, and 5) are plotted against the actual tumor volumes in plot 610 of FIG. 6B. As illustrated, the comparison illustrates that the system and method embodiments described herein outperform the conventional technology.

Further, predictive performance of the tumor dynamics prediction system with and without the heterogeneous graph encoder was computed for different observations windows with respect to R2 with the mean R2 and standard deviation over 5-fold cross-validation reported below:

TABLE 2

| Observation | Without Heterogeneous | With Heterogeneous |
|---|---|---|
| Window | Graph Encoder | Graph Encoder |
| 7 | %23.3 ± 5.2 | %30.2 ± 4.9 |
| 14 | %45.6 ± 4.8 | %47.9 ± 4.7 |
| 21 | %58.6 ± 4.1 | %60.8 ± 4.2 |
| 28 | %65.2 ± 3.9 | %65.9 ± 3.8 |

The heterogeneous graph encoder (e.g., heterogeneous graph encoder 124 described with respect to FIG. 1 and FIG. 2) includes a graph convolutional network (GCN) system. The GCN system may be pretrained. For example, the GCN may be initialized based on a variational graph auto-encoder (e.g., VGAE 218 in FIG. 2) that is pretrained using the RNA sequence data for the PDX models in the dataset. Each PDX model was represented as a graph with genes as the nodes of the graph. Tumor-specific graphs were obtained from TissueNexus. Model performance was assessed where the GCN system was used without pretraining and with pretraining. The pretrained GCN system provided improved model performance with about 9% improvement in F1 score (e.g., mean of precision and recall) and 5% improvement in AUROC (area under receiver operating characteristics curve).

Model performance was also assessed with respect to prediction of mRECIST categories for treatment response. The performance of models that included the heterogeneous graph encoder outperformed models without the heterogeneous graph encoder.

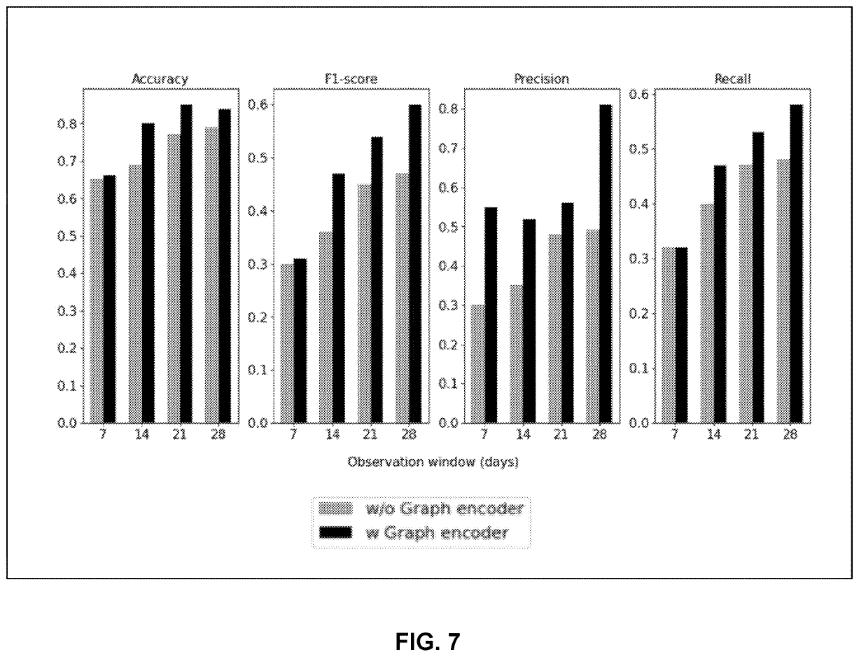

FIG. 7 is an illustration of four different graphs comparing the performance of a tumor dynamics prediction system when a heterogeneous graph encoder is used and when the heterogeneous graph encoder is not used in accordance with one or more embodiments. The two different prediction systems (with and without the heterogeneous graph encoder) were compared with respect to their performance in predicting mRECIST categories for treatment response. As depicted, the tumor dynamics prediction systems with the heterogeneous graph encoder had improved performance with respect to accuracy, F1 score, precision, and recall.

### V. Example Computing System

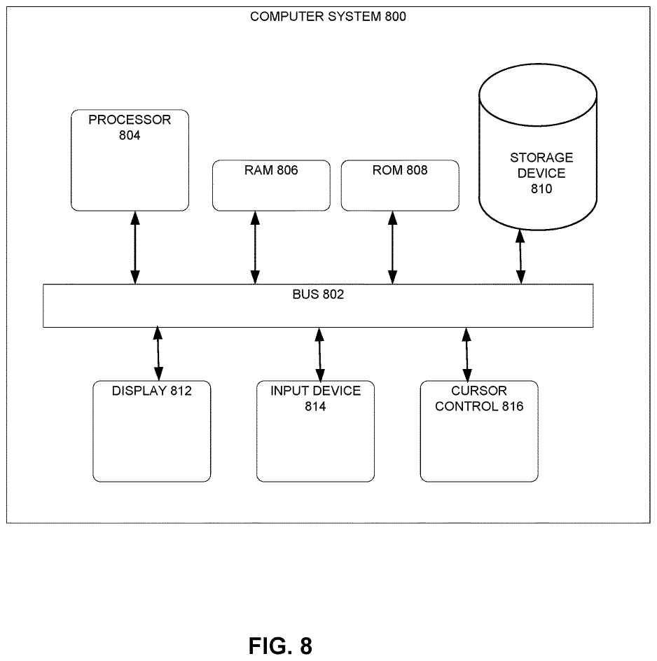

FIG. 8 is a block diagram illustrating an example of a computing system, in accordance with one or more example embodiments. Computing system 800 may be used to implement computing platform 106 in FIG. 1 and/or any components therein.

In one or more examples, computer system 800 can include a bus 802 or other communication mechanism for communicating information, and a processor 804 coupled with bus 802 for processing information. In various embodiments, computer system 800 can also include a memory, which can be a random-access memory (RAM) 806 or other dynamic storage device, coupled to bus 802 for determining instructions to be executed by processor 804. Memory also can be used for storing temporary variables or other intermediate information during execution of instructions to be executed by processor 804. In various embodiments, computer system 800 can further include a read only memory (ROM) 808 or other static storage device coupled to bus 802 for storing static information and instructions for processor 804. A storage device 810, such as a magnetic disk or optical disk, can be provided and coupled to bus 802 for storing information and instructions.

In various embodiments, computer system 800 can be coupled via bus 802 to a display 812, such as a cathode ray tube (CRT) or liquid crystal display (LCD), for displaying information to a computer user. An input device 814, including alphanumeric and other keys, can be coupled to bus 802 for communicating information and command selections to processor 804. Another type of user input device is a cursor control 816, such as a mouse, a joystick, a trackball, a gesture input device, a gaze-based input device, or cursor direction keys for communicating direction information and command selections to processor 804 and for controlling cursor movement on display 812. This input device 814 typically has two degrees of freedom in two axes, a first axis (e.g., x) and a second axis (e.g., y), that allows the device to specify positions in a plane. However, it should be understood that input devices 814 allowing for three-dimensional (e.g., x, y, and z) cursor movement are also contemplated herein.

Consistent with certain implementations of the present teachings, results can be provided by computer system 800 in response to processor 804 executing one or more sequences of one or more instructions contained in RAM 806. Such instructions can be read into RAM 806 from another computer-readable medium or computer-readable storage medium, such as storage device 810. Execution of the sequences of instructions contained in RAM 806 can cause processor 804 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 804 for execution. Such a medium can take many forms, including but not limited to, non-volatile media, volatile media, and transmission media. Examples of non-volatile media can include, but are not limited to, optical, solid state, magnetic disks, such as storage device 810. Examples of volatile media can include, but are not limited to, dynamic memory, such as RAM 806. Examples of transmission media can include, but are not limited to, coaxial cables, copper wire, and fiber optics, including the wires that comprise bus 802.

Common forms of computer-readable media include, for example, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 804 of computer system 800 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 800, whereby processor 804 would execute the analyses and determinations provided by these engines, subject to instructions provided by any one of, or a combination of, the memory components RAM 806, ROM, 808, or storage device 810 and user input provided via input device 814.

In some example embodiments, the computing system 800 can be used to execute various interactive computer software applications that can be used for organization, analysis, and/or storage of data in various formats. Alternatively, the computing system 800 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 814. The user interface can be generated and presented to a user by the computing system 800 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus, and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

### VI. Example Definitions and Context

The disclosure is not limited to these example embodiments and applications or to the manner in which the example embodiments and applications operate or are described herein. Moreover, the figures may show simplified or partial views, and the dimensions of elements in the figures may be exaggerated or otherwise not in proportion.

Where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology are described herein are those well-known and commonly used in the art.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person or animal undergoing treatment, a person or animal undergoing anti-cancer therapies, a person or animal being monitored for remission or recovery, a person or animal undergoing a preventative health analysis (e.g., due to their medical history), or any other person or patient or animal of interest. In various cases, “subject” and “patient” may be used interchangeably herein.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “about” used with respect to numerical values or parameters or characteristics that can be expressed as numerical values means within ten percent of the numerical values. For example, “about 50” means a value in the range from 45 to 55, inclusive.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning (ML) algorithms, or a combination thereof.

As used herein, “machine learning” may include the practice of using algorithms to parse data, learn from it, and then make a determination or prediction about something in the world. Machine learning uses algorithms that can learn from data without relying on rules-based programming.

As used herein, an “artificial neural network” or “neural network” may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in, for example, two ways; when it is being trained (e.g., using a training dataset) it is in training mode and when it puts what it has learned into practice (e.g., using a test dataset) it is in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may learn by being fed training data (learning examples) and eventually learns how to reach the correct output, even when it is presented with a new range or set of inputs.

A neural network may process information in two ways; when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Graph Convolutional Network (GCN), a Residual Neural Network (ResNet), an Ordinary Differential Equations Neural Networks (neural-ODE), or another type of neural network.

With a neural-ODE, the derivative of the hidden state may be parameterized using a neural network. The neural-ODE may be capable of incorporating data for arbitrary times into a continuous time-series (or time course).

As used herein, an “encoder” may refer to a type of neural network that learns to encode (e.g., efficiently encode) a set of data into a vector of parameters having a number of dimensions. The number of dimensions may be preselected.

As used herein, “an ordinary differential equations (ODE) module” may refer to a neural network architecture that includes at least one neural network. The at least one neural network may include, for example, at least one recurrent neural network, at least one neural-ODE solver, or a combination thereof.

As used herein, “deep learning” may refer to the use of multi-layered artificial neural networks to automatically learn representations from input data such as images, video, text, etc., without human provided knowledge, to deliver highly accurate predictions in tasks such as object detection/identification, speech recognition, language translation, etc.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology, and toxicology are described herein are those well-known and commonly used in the art.

### VII. Recitation of Embodiments

Embodiment 1: A method of predicting future tumor data, the method comprising: receiving a tumor embedding at a neural ordinary differential equations (ODE) system; receiving a graph embedding at the neural ODE system, wherein the graph embedding fuses drug information, disease information, and gene relationship information; generating a predicted tumor size at a future point in time using the tumor embedding, the graph embedding, and the neural ODE system; and administering a candidate tumor treatment to a subject in response to the predicted tumor size being less than a selected threshold.

Embodiment 2: The method of Embodiment 1, further comprising: generating the tumor embedding using a tumor encoder and observed tumor data that is observed over a selected period of time after a reference point in time, wherein the selected period of time is a number of days between 2 days and 45 days.

Embodiment 3: The method of Embodiment 2, wherein the tumor encoder comprises a recurrent neural network and wherein the observed tumor data includes observed tumor volume.

Embodiment 4: The method of any one of Embodiments 1-3, further comprising: generating the graph embedding using a heterogeneous graph encoder that fuses together at least two or more embeddings generated using bipartite graph convolution attention networks.

Embodiment 5: The method of any one of Embodiments 1-4, further comprising; applying a graph convolution network system to a gene domain of a heterogeneous graph to generate a gene-gene embedding; applying a first bipartite graph convolutional network system to a drug domain and a gene domain of the heterogeneous graph to generate a drug-gene embedding, wherein the first bipartite graph convolutional network system includes a bipartite graph attention convolution layer; applying a second bipartite graph convolutional network system to a disease domain and a gene domain of the heterogeneous graph to generate a disease-gene embedding, wherein the first bipartite graph convolutional network system includes a bipartite graph attention convolution layer; and forming the graph embedding using the gene-gene embedding, the drug-gene embedding, and the disease-gene embedding.

Embodiment 6: The method of Embodiment 5, wherein forming the graph embedding using the gene-gene embedding, the drug-gene embedding, and the disease-gene embedding comprises: concatenating the gene-gene embedding, the drug-gene embedding, and the disease-gene embedding to form the graph embedding.

Embodiment 7: The method of Embodiment 5, wherein the graph convolution network system is initialized using a pretrained variational graph auto-encoder (VAGE).

Embodiment 8: The method of Embodiment 7, wherein the variational graph auto-encoder is pretrained using RNA sequence data from a training dataset that includes data for a plurality of patient-derived xenograft (PDX) models.

Embodiment 9: The method of any one of Embodiments 1-8, wherein the candidate tumor treatment comprises at least one of clofarabine, cimetidine, thiamine, or arsenic trioxide or wherein the candidate tumor treatment comprises at least one of dextromethorphan, solifencacin, atomoxetine, venlafaxine, or tapentadol.

Embodiment 10: The method of any one of Embodiments 1-9, wherein the candidate tumor treatment comprises at least one a small molecule inhibitor of SHP2, a neoantigen cancer vaccine, a T-cell therapy, a personalized cancer vaccine, a neoantigen-directed T cell therapy, an immunotherapeutic, alectinib, bevacizumab, glofitamab-gxbm, cobimetinib, vismodegib, obinutuzumab, trastuzumab, hyaluronidase (e.g., hyaluronidase-oysk, hyaluronidase-zzxf, hyaluronidase human), ado-trastuzumab emtansine, mosunetuzumab-axgb, pertuzumab, polatuzumab vedotin-piiq, rituximab, entrectinib, erlotinib, atezolizumab, venetoclax, capecitabine, or vemurafenib.

Embodiment 11: A method of predicting future tumor data, the method comprising: generating a tumor embedding using a tumor encoder and observed tumor data; generating a graph embedding using a heterogeneous graph encoder and a heterogeneous graph that includes nodes in a gene domain, a drug domain, and a disease domain; generating a predicted tumor size at a future point in time using the tumor embedding, the graph embedding, and a neural ordinary differential equations (ODE) system; and adjusting a treatment regimen for a treatment to be administered to a subject based on the predicted tumor size.

Embodiment 12: The method of Embodiment 11, wherein the tumor encoder includes a recurrent neural network and wherein generating the tumor embedding comprises: applying the recurrent neural network to the observed tumor data to generate the tumor embedding.

Embodiment 13: The method of any one of Embodiments 11-12, wherein generating the graph embedding comprises: applying a graph convolution network system to the gene domain of the heterogeneous graph to generate a gene-gene embedding; applying a first bipartite graph convolutional network system to project the drug domain onto the gene domain of the heterogeneous graph to generate a drug-gene embedding, wherein the first bipartite graph convolutional network system includes a bipartite graph attention convolution layer; applying a second bipartite graph convolutional network system to project the gene domain onto the disease domain of the heterogeneous graph to generate a disease-gene embedding, wherein the first bipartite graph convolutional network system includes a bipartite graph attention convolution layer; and forming the graph embedding using the gene-gene embedding, the drug-gene embedding, and the disease-gene embedding.

Embodiment 14: The method of any one of Embodiments 11-13, wherein the treatment comprises at least one of clofarabine, cimetidine, thiamine, or arsenic trioxide or wherein the treatment comprises at least one of, dextromethorphan, solifencacin, atomoxetine, venlafaxine, or tapentadol.

Embodiment 15: The method of any one of Embodiments 11-14, further comprising: predicting a treatment response classification based on the predicted tumor size.

Embodiment 16: A system comprising: one or more data processors; and a non-transitory computer readable storage medium containing instructions which, when executed on the one or more data processors, cause the one or more data processors to: receive a tumor embedding at a neural ordinary differential equations (ODE) system; receive a graph embedding at the neural ODE system, wherein the graph embedding fuses drug information, disease information, and gene relationship information; generate a predicted tumor size at a future point in time using the tumor embedding, the graph embedding, and the neural ODE system; and administer a candidate tumor treatment to a subject in response to the predicted tumor size being less than a selected threshold.

Embodiment 17: The system of Embodiment 16, further comprising: generate the tumor embedding using a tumor encoder and observed tumor data that is observed over a selected period of time after a reference point in time, wherein the selected period of time is a number of days between 2 days and 45 days.

Embodiment 18: The system of Embodiment 17, wherein the tumor encoder comprises a recurrent neural network and wherein the observed tumor data includes observed tumor volume.

Embodiment 19: The system of any one of Embodiments 16-18, further comprising:
generate the graph embedding using a heterogeneous graph encoder that fuses together at least two or more embeddings generated using bipartite graph convolution attention networks.

Embodiment 20: The system of any one of Embodiments 16-19, further comprising;
apply a graph convolution network system to a gene domain of a heterogeneous graph to generate a gene-gene embedding; apply a first bipartite graph convolutional network system to a drug domain and a gene domain of the heterogeneous graph to generate a drug-gene embedding, wherein the first bipartite graph convolutional network system includes a bipartite graph attention convolution layer; apply a second bipartite graph convolutional network system to a disease domain and a gene domain of the heterogeneous graph to generate a disease-gene embedding, wherein the first bipartite graph convolutional network system includes a bipartite graph attention convolution layer; and form the graph embedding using the gene-gene embedding, the drug-gene embedding, and the disease-gene embedding.

Embodiment 21: The system of Embodiment 20, wherein formation of the graph embedding using the gene-gene embedding, the drug-gene embedding, and the disease-gene embedding comprises: concatenate the gene-gene embedding, the drug-gene embedding, and the disease-gene embedding to form the graph embedding.

Embodiment 22: The system of Embodiment 20, wherein the graph convolution network system is initialized using a pretrained variational graph auto-encoder (VAGE).

Embodiment 23: The system of Embodiment 22, wherein the variational graph auto-encoder is pretrained using RNA sequence data from a training dataset that includes data for a plurality of patient-derived xenograft (PDX) models.

Embodiment 24: The system of any one of Embodiments 16-23, wherein the candidate tumor treatment comprises at least one of clofarabine, cimetidine, thiamine, or arsenic trioxide or wherein the candidate tumor treatment comprises at least one of dextromethorphan, solifencacin, atomoxetine, venlafaxine, or tapentadol.

Embodiment 25: The system of any one of Embodiments 16-23, wherein the candidate tumor treatment comprises at least one of a small molecule inhibitor of SHP2, a neoantigen cancer vaccine, a T-cell therapy, a personalized cancer vaccine, a neoantigen-directed T cell therapy, an immunotherapeutic, alectinib, bevacizumab, glofitamab-gxbm, cobimetinib, vismodegib, obinutuzumab, trastuzumab, hyaluronidase (e.g., hyaluronidase-oysk, hyaluronidase-zzxf, hyaluronidase human), ado-trastuzumab emtansine, mosunetuzumab-axgb, pertuzumab, polatuzumab vedotin-piiq, rituximab, entrectinib, erlotinib, atezolizumab, venetoclax, capecitabine, or vemurafenib.

Embodiment 26: A system comprising: one or more data processors; and a non-transitory computer readable storage medium containing instructions which, when executed on the one or more data processors, cause the one or more data processors to: generate a tumor embedding using a tumor encoder and observed tumor data; generate a graph embedding using a heterogeneous graph encoder and a heterogeneous graph that includes nodes in a gene domain, a drug domain, and a disease domain; generate a predicted tumor size at a future point in time using the tumor embedding, the graph embedding, and a neural ordinary differential equations (ODE) system; and adjust a treatment regimen for a treatment to be administered to a subject based on the predicted tumor size.

Embodiment 27: The system of Embodiment 26, wherein the tumor encoder includes a recurrent neural network and wherein generation of the tumor embedding comprises: apply the recurrent neural network to the observed tumor data to generate the tumor embedding.

Embodiment 28: The system of any one of Embodiments 26-27, wherein generation of the graph embedding comprises: apply a graph convolution network system to the gene domain of the heterogeneous graph to generate a gene-gene embedding; apply a first bipartite graph convolutional network system to project the drug domain onto the gene domain of the heterogeneous graph to generate a drug-gene embedding, wherein the first bipartite graph convolutional network system includes a bipartite graph attention convolution layer; apply a second bipartite graph convolutional network system to project the gene domain onto the disease domain of the heterogeneous graph to generate a disease-gene embedding, wherein the first bipartite graph convolutional network system includes a bipartite graph attention convolution layer; and form the graph embedding using the gene-gene embedding, the drug-gene embedding, and the disease-gene embedding.

Embodiment 29: The system of any one of Embodiments 26-28, wherein the treatment comprises at least one of clofarabine, cimetidine, thiamine, or arsenic trioxide or wherein the treatment comprises at least one of, dextromethorphan, solifencacin, atomoxetine, venlafaxine, or tapentadol.

Embodiment 29: The system of any one of Embodiments 26-28, wherein the treatment comprises at least one of a small molecule inhibitor of SHP2, a neoantigen cancer vaccine, a T-cell therapy, a personalized cancer vaccine, a neoantigen-directed T cell therapy, an immunotherapeutic, alectinib, bevacizumab, glofitamab-gxbm, cobimetinib, vismodegib, obinutuzumab, trastuzumab, hyaluronidase (e.g., hyaluronidase-oysk, hyaluronidase-zzxf, hyaluronidase human), ado-trastuzumab emtansine, mosunetuzumab-axgb, pertuzumab, polatuzumab vedotin-piiq, rituximab, entrectinib, erlotinib, atezolizumab, venetoclax, capecitabine, or vemurafenib.

Embodiment 31: A system comprising: one or more data processors; and a non-transitory computer readable storage medium containing instructions which, when executed on the one or more data processors, cause the one or more data processors to perform part or all of one or more methods disclosed in Embodiments 1-15.

Embodiment 32: A computer-program product tangibly embodied in a non-transitory machine-readable storage medium, including instructions configured to cause one or more data processors to perform part or all of one or more methods disclosed in Embodiments 1-15.

### VIII. Additional Considerations

The headers and subheaders between sections and subsections of this document are included solely for the purpose of improving readability and do not imply that features cannot be combined across sections and subsection. Accordingly, sections and subsections do not describe separate embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the disclosure. Thus, it should be understood that although the present disclosure has been specifically disclosed by embodiments and optional features, modification and variation of the concepts herein disclosed may be resorted to by those skilled in the art, and that such modifications and variations are considered to be within the scope of this disclosure as defined by the appended claims.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Further, the subject matter described herein can be embodied in systems, apparatus, methods, and/or articles depending on the desired configuration. The implementations set forth in the description herein do not represent all implementations consistent with the subject matter described. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method of predicting future tumor data, the method comprising:
receiving a tumor embedding at a neural ordinary differential equations (ODE) system;
receiving a graph embedding at the neural ODE system, wherein the graph embedding fuses drug information, disease information, and gene relationship information;
generating a predicted tumor size at a future point in time using the tumor embedding, the graph embedding, and the neural ODE system; and
administering a candidate tumor treatment to a subject in response to the predicted tumor size being less than a selected threshold.

2. The method of claim 1, further comprising:
generating the tumor embedding using a tumor encoder and observed tumor data that is observed over a selected period of time after a reference point in time, wherein the selected period of time is a number of days between 2 days and 45 days.

3. The method of claim 2, wherein the tumor encoder comprises a recurrent neural network and wherein the observed tumor data includes observed tumor volume.

4. The method of claim 1, further comprising:
generating the graph embedding using a heterogeneous graph encoder that fuses together at least two or more embeddings generated using bipartite graph convolution attention networks.

5. The method of claim 1, further comprising;
applying a graph convolution network system to a gene domain of a heterogeneous graph to generate a gene-gene embedding;
applying a first bipartite graph convolutional network system to a drug domain and a gene domain of the heterogeneous graph to generate a drug-gene embedding, wherein the first bipartite graph convolutional network system includes a bipartite graph attention convolution layer;
applying a second bipartite graph convolutional network system to a disease domain and a gene domain of the heterogeneous graph to generate a disease-gene embedding, wherein the first bipartite graph convolutional network system includes a bipartite graph attention convolution layer; and
forming the graph embedding using the gene-gene embedding, the drug-gene embedding, and the disease-gene embedding.

6. The method of claim 5, wherein forming the graph embedding using the gene-gene embedding, the drug-gene embedding, and the disease-gene embedding comprises:
concatenating the gene-gene embedding, the drug-gene embedding, and the disease-gene embedding to form the graph embedding.

7. The method of claim 5, wherein the graph convolution network system is initialized using a pretrained variational graph auto-encoder (VAGE).

8. The method of claim 7, wherein the variational graph auto-encoder is pretrained using RNA sequence data from a training dataset that includes data for a plurality of patient-derived xenograft (PDX) models.

9. The method of claim 1, wherein the candidate tumor treatment comprises at least one of clofarabine, cimetidine, thiamine, or arsenic trioxide, or wherein the candidate tumor treatment comprises at least one of dextromethorphan, solifencacin, atomoxetine, venlafaxine, or tapentadol.

10. A method of predicting future tumor data, the method comprising:
generating a tumor embedding using a tumor encoder and observed tumor data;
generating a graph embedding using a heterogeneous graph encoder and a heterogeneous graph that includes nodes in a gene domain, a drug domain, and a disease domain;
generating a predicted tumor size at a future point in time using the tumor embedding, the graph embedding, and a neural ordinary differential equations (ODE) system; and
adjusting a treatment regimen for a treatment to be administered to a subject based on the predicted tumor size.

11. The method of claim 10, wherein the tumor encoder includes a recurrent neural network and wherein generating the tumor embedding comprises:
applying the recurrent neural network to the observed tumor data to generate the tumor embedding.

12. The method of claim 10, wherein generating the graph embedding comprises:
applying a graph convolution network system to the gene domain of the heterogeneous graph to generate a gene-gene embedding;
applying a first bipartite graph convolutional network system to project the drug domain onto the gene domain of the heterogeneous graph to generate a drug-gene embedding, wherein the first bipartite graph convolutional network system includes a bipartite graph attention convolution layer;
applying a second bipartite graph convolutional network system to project the gene domain onto the disease domain of the heterogeneous graph to generate a disease-gene embedding, wherein the first bipartite graph convolutional network system includes a bipartite graph attention convolution layer; and
forming the graph embedding using the gene-gene embedding, the drug-gene embedding, and the disease-gene embedding.

13. The method of claim 10, wherein the treatment comprises at least one of clofarabine, cimetidine, thiamine, or arsenic trioxide or wherein the treatment comprises at least one of dextromethorphan, solifencacin, atomoxetine, venlafaxine, or tapentadol.

14. The method of claim 10, further comprising:
predicting a treatment response classification based on the predicted tumor size.

15. A system comprising:
one or more data processors; and
a non-transitory computer readable storage medium containing instructions which, when executed on the one or more data processors, cause the one or more data processors to:
receive a tumor embedding at a neural ordinary differential equations (ODE) system;
receive a graph embedding at the neural ODE system, wherein the graph embedding fuses drug information, disease information, and gene relationship information;
generate a predicted tumor size at a future point in time using the tumor embedding, the graph embedding, and the neural ODE system; and
administer a candidate tumor treatment to a subject in response to the predicted tumor size being less than a selected threshold.

16. The system of claim 15, further comprising:
generate the tumor embedding using a tumor encoder and observed tumor data that is observed over a selected period of time after a reference point in time, wherein the selected period of time is a number of days between 2 days and 45 days.

17. The system of claim 16, wherein the tumor encoder comprises a recurrent neural network and wherein the observed tumor data includes observed tumor volume.

18. The system of claim 15, further comprising:
generate the graph embedding using a heterogeneous graph encoder that fuses together at least two or more embeddings generated using bipartite graph convolution attention networks.

19. The system of claim 15, further comprising;
apply a graph convolution network system to a gene domain of a heterogeneous graph to generate a gene-gene embedding;
apply a first bipartite graph convolutional network system to a drug domain and a gene domain of the heterogeneous graph to generate a drug-gene embedding, wherein the first bipartite graph convolutional network system includes a bipartite graph attention convolution layer;
apply a second bipartite graph convolutional network system to a disease domain and a gene domain of the heterogeneous graph to generate a disease-gene embedding, wherein the first bipartite graph convolutional network system includes a bipartite graph attention convolution layer; and
form the graph embedding using the gene-gene embedding, the drug-gene embedding, and the disease-gene embedding.

20. The system of claim 19, wherein formation of the graph embedding using the gene-gene embedding, the drug-gene embedding, and the disease-gene embedding comprises:
concatenate the gene-gene embedding, the drug-gene embedding, and the disease-gene embedding to form the graph embedding.

21. The system of claim 19, wherein the graph convolution network system is initialized using a pretrained variational graph auto-encoder (VAGE).

22. The system of claim 21, wherein the variational graph auto-encoder is pretrained using RNA sequence data from a training dataset that includes data for a plurality of patient-derived xenograft (PDX) models.

23. The system of claim 15, wherein the candidate tumor treatment comprises at least one of clofarabine, cimetidine, thiamine, or arsenic trioxide or wherein the treatment comprises at least one of dextromethorphan, solifencacin, atomoxetine, venlafaxine, or tapentadol.

24. A system comprising:
one or more data processors; and
a non-transitory computer readable storage medium containing instructions which, when executed on the one or more data processors, cause the one or more data processors to:
generate a tumor embedding using a tumor encoder and observed tumor data;
generate a graph embedding using a heterogeneous graph encoder and a heterogeneous graph that includes nodes in a gene domain, a drug domain, and a disease domain;
generate a predicted tumor size at a future point in time using the tumor embedding, the graph embedding, and a neural ordinary differential equations (ODE) system; and
adjust a treatment regimen for a treatment to be administered to a subject based on the predicted tumor size.

25. The system of claim 24, wherein the tumor encoder includes a recurrent neural network and wherein generation of the tumor embedding comprises:
apply the recurrent neural network to the observed tumor data to generate the tumor embedding.

26. The system of claim 24, wherein generation of the graph embedding comprises:
apply a graph convolution network system to the gene domain of the heterogeneous graph to generate a gene-gene embedding;
apply a first bipartite graph convolutional network system to project the drug domain onto the gene domain of the heterogeneous graph to generate a drug-gene embedding, wherein the first bipartite graph convolutional network system includes a bipartite graph attention convolution layer;
apply a second bipartite graph convolutional network system to project the gene domain onto the disease domain of the heterogeneous graph to generate a disease-gene embedding, wherein the first bipartite graph convolutional network system includes a bipartite graph attention convolution layer; and
form the graph embedding using the gene-gene embedding, the drug-gene embedding, and the disease-gene embedding.

27. The system of claim 24, wherein the treatment comprises at least one of clofarabine, cimetidine, thiamine, or arsenic trioxide or wherein the treatment comprises at least one of dextromethorphan, solifencacin, atomoxetine, venlafaxine, or tapentadol.
